(19) United States
(12) Patent Application Publication
(10) Pub. No.: US 2017/0369398 A1
(43) Pub. Date: Dec. 28, 2017

(54) PROCESS FOR DEHYDROGENATION OF ALKYL-CONTAINING COMPOUNDS USING MOLYBDENUM AND TUNGSTEN NITROSYL COMPLEXES

(71) Applicants: Dow Global Technologies LLC, Midland, MI (US); University of British Columbia, Vancouver (CA)

(72) Inventors: Peter Legzdins, West Vancouver, BC (CA); Rhett Baillie, Vancouver, BC (CA); Russ Wakeham, Vancouver, BC (CA); Devon C. Rosenfeld, Freeport, TX (US)

(73) Assignees: Dow Global Technologies LLC, Midland, MI (US); University of British Columbia, Vancouver, BC (CA)

(21) Appl. No.: 15/541,191

(22) PCT Filed: Jan. 8, 2016
(86) PCT No.: PCT/US2016/012562

§ 371 Date: Jun. 30, 2017

Related U.S. Application Data
(60) Provisional application No. 62/101,413, filed on Jan. 9, 2015.

(51) Int. Cl. C07C 5/333; B01J 31/22

(52) U.S. Cl. CPC C07C 5/333; B01J 31/2295; B01J 2531/66; B01J 2231/766; C07C 2531/22; B01J 2531/64

(57) ABSTRACT

A process for the dehydrogenation of alkyl-containing compounds comprises reacting an alkyl-containing compound and a Group VI nitrosyl complex characterized as a transition metal complex having the composition Cp′M(NO)(R1)(R2), wherein Cp′ is selected from certain substituted and unsubstituted η⁵-cyclopentadienyl groups; M is W or Mo; and R1 and R2 are independently selected from CH₂C(CH₃)₃; CH₂Si(CH₃)₃; CH₂(C₆H₅); CH₃; hydrogen; and η³-allyl; provided that if R1 is hydrogen, R2 is η³-allyl; under conditions such that the alkyl-containing compound is converted to an olefin, and in particular embodiments, a terminal olefin. The dehydrogenation can be carried out using a neat and/or undried alkyl-containing compound and/or may be conducted under air, and does not require a sacrificial olefin to drive the reaction, thereby increasing convenience and decreasing cost in comparison with some other dehydrogenation processes.

This patent application claims the benefit of U.S. Patent Application Ser. No. 62/101,413, filed Jan. 9, 2015, entitled “PROCESS FOR DEHYDROGENATION OF ALKYL-CONTAINING COMPOUNDS USING MOLYBDENUM AND TUNGSTEN NITROSYL COMPLEXES,” which is incorporated herein by reference in its entirety.

The invention relates to processes to dehydrogenate alkyl-containing compounds. More particularly, it relates to a process that selectively dehydrogenates alkyl-containing compounds to form olefins.

Over the last several years industry has been challenged by an ever-increasing need to develop new, and better, methods to carry out dehydrogenation and thereby increase substrate unsaturation. Such dehydrogenations may find particular use in a variety of processes to convert natural gas components, such as ethane and propane, to more industrially useful compounds, such as olefins.

Terminal olefins are of greatest use in the chemical and petrochemical industry therefore dehydrogenation processes selective to terminal olefins are of tremendous interest. Generally some type of catalyst is necessary, since non-catalytic dehydrogenations normally occur only at very high temperature.

The generation of olefins from alkyl-containing compounds via catalytic dehydrogenation has been studied using organometallic complexes in the homogenous phase. For example, WO 2013/052253 A1 (Goldman, et al.) discusses iridium catalyst complexes useful for alkane dehydrogenation. The iridium complexes are ligated by benzimidazolyl-containing ligands forming NCN pincer complexes. These iridium complexes are used as catalysts for the conversion of alkanes to olefins in the presence or absence of a hydrogen acceptor. U.S. Pat. No. 5,780,701 (Kaska, et al.) discusses a process to convert alkanes to alkenes using a catalyst including ruthenium, rhodium, palladium, osmium, iridium or platinum. WO 2002/085920 A2 discloses alkane dehydrogenation using a catalyst including a first transition metal, a second transition metal π-bonded to an η⁵-aromatic ligand, and a pincer ligand. U.S. Patent Publication 2004/0181104 A1 (Yeh, et al.) discloses a dehydrogenation catalyst including an organometallic pincer complex bonded to a mesoporous inorganic oxide support.

Additional dehydrogenation research is disclosed in, for example, Gupta, M.; Hagen, C.; Kaska, W. C.; Kramer, R. E.; Jensen, C. M. “Catalytic Dehydrogenation of Cycloalkanes to Arenes by a Dihydrido Iridium P—C—P Pincer Complex,” J. Am. Chem. Soc. 1997, 119, 840-841, which reports transfer dehydrogenation of cycloalkanes using IrH₂{C₆H₃-2,6-(CH₂PBu^{t}₂)₂}. See, also, e.g., Xu, W.; Rosini. G. P.; Gupta, M.; Jensen, C. M.; Kaska, W. C.; Krogh-Jespersen, K.; Goldman, A. S. “Thermochemical Alkane Dehydrogenation Catalyzed in Solution Without the Use of a Hydrogen Acceptor,” J. Chem. Soc., Chem. Comm. 1997, 2273-2274, which reports alkane dehydrogenation catalyzed by IrH₂{C₆H₃-2,6-(CH₂PBu^{t}₂)₂}. Additional information is available in, for example, Morales-Morales, D.; Lee, D. W.; Wang, Zhaohui; Jensen, C. M. “Oxidative Addition of Water by an Iridium PCP Pincer Complex: Catalytic Dehydrogenation of Alkanes by IrH(OH){C₆H₃-2,6-(CH₂PBu^{t}₂)₂},” Organometallics 2001, 20, 1144-1147; and Lee, D. W.; Kaska, W. C.; Jensen, C. M. “Mechanistic Features of Iridium Pincer Complex Catalyzed Hydrocarbon Dehydrogenation Reactions: Inhibition upon Formation of a p-Dinitrogen Complex,” Organometallics 1998, 17, 1-3. Further discussion of the Ir(pincer) catalysts may also be found in, e.g., Williams, D. B.; Kaminsky, W.; Mayer, J. M.; Goldberg, K. I. “Reactions of Iridium Hydride Pincer Complexes with Dioxygen: New Dioxygen Complexes and Reversible O₂ Binding,” Chem. Comm. 2008, 35, 4195-4197. In general, the aforementioned literature publications highlight the various limitations of these pincer complexes, including, for example, that they may decompose in the presence of water and oxygen; require a costly noble metal (iridium) to prepare; are generally not selective to produce a specifically terminal olefin; may need to employ a hydrogen acceptor, such as t-butylethylene; and/or may undesirably react with nitrogen.

In view of the above, there remains a need for new processes that can effectively dehydrogenate suitable substrates, such as but not necessarily limited to alkanes, to form olefins, and in particular embodiments, terminal olefins. Desirably these processes can be carried out in the presence of air and water, in the presence of heteroatom-containing functional groups (halogens, nitrogen, oxygen and sulfur containing compounds), and in the absence of a hydrogen acceptor, such as t-butylethylene. Such processes would offer both desirable industrial applicability and improved economics.

In one aspect the invention provides a process for a dehydrogenation, comprising reacting an alkyl-containing compound and a transition metal complex having the composition Cp′M(NO)(R1)(R2) wherein Cp′ is a substituted or unsubstituted η⁵-cyclopentadienyl group, wherein the substituents are independently selected from hydrogen and moieties containing from 1 to 40 non-hydrogen atoms selected from hydrocarbyl, arylalkyl, hydrocarbylsilyl, halo-substituted hydrocarbyl, hydrocarbyloxy-substituted hydrocarbyl, hydrocarbylamino-substituted hydrocarbyl, hydrocarbylsilyl-substituted hydrocarbyl, hydrocarbylamino, di(hydrocarbyl)amino, and hydrocarbyloxy moieties, and combinations thereof; and wherein M is selected from W and Mo; and wherein R1 and R2 are each independently selected from (a) CH₂C(CH₃)₃; (b) CH₂Si(CH₃)₃; (c) CH₂(C₆H₅); (d) CH₃; (e) hydrogen; and (f) η³-allyl; provided that if R1 is hydrogen, R2 is η³-allyl; under conditions such that the alkyl-containing compound is converted to an olefin.

The present invention provides a process for dehydrogenations to form olefins, and in particular terminal olefins, which process may be readily conducted, where desired or convenient, in the presence of water and/or air. It is particularly useful for dehydrogenations of any alkyl-containing compounds, which are defined as compounds that include adjacent single-bonded carbon atoms possessing at least one hydrogen atom each. Such may include compounds having carbon numbers ranging from 2 to 20 carbon atoms, preferably from 2 to 12 carbon atoms, and more preferably from 2 to 10 carbon atoms. Included among these, in non-limiting example, are linear alkanes, such as ethane, propane, butane, pentane, hexane, heptane, octane, nonane, decane, undecane, dodecane, and combinations thereof. Their specifically linear n-isomers as well as other isomers may be included. Also included in non-limiting example are corresponding and related cyclic and non-cyclic compounds, such as ethylcyclohexane; ethylbenzene; propylbenzene; alkyl halides (alternatively termed halogenated alkanes) such as 1-chlorobutane; ethers such as di-n-butyl ether; and nitriles such as propionitrile. By definition any of these compounds may be dehydrogenated to form adjacent double-bonded carbons, i.e., an olefin. In preferred embodiments, such dehydrogenation results in a terminal olefin, i.e., one of the adjacent double-bonded carbons is in a terminal position.

The transition metal complex composition used herein is defined as an organometallic compound wherein the metal is either tungsten (W) or molybdenum (Mo). In certain particular embodiments W is preferred. This organometallic transition metal complex has the chemical composition Cp′M(NO)(R1)(R2) wherein Cp′ is a substituted or unsubstituted η⁵-cyclopentadienyl group, wherein the substituent is as described hereinbelow; wherein M is selected from tungsten (W) and molybdenum (Mo); and wherein R1 and R2 are each independently selected from (a) CH₂C(CH₃)₃; (b) CH₂Si(CH₃)₃; (c) CH₂(C₆H₅); (d) CH₃; (e) hydrogen; and (f) η³-allyl; provided that if R1 is hydrogen, R2 is η³-allyl. It is noted that, because selection is “independent,” R1 and R2 may be the same or different, except that the proviso stating that “if R1 is hydrogen, R2 is η³-allyl” must be applied. “(NO)” is a nitrosyl group.

The η³-allyl is an allyl ligand selected from: (a) η³—CₙH₍₂ₙ₋₁₎, (b) η³—CH₂CH(CH₃)₂; (c) η³—CH₂CHCHSi(CH₃)₃; (d) η³—CH₂CHCH(C₆H₅); and (e) CH(C₆H₅)CHCH(C₆H₅); wherein n is an integer from 3 to 10.

Formula A hereinbelow is a structural formula of the Cp′M(NO)(R1)(R2) molecule that is the transition metal complex used in the present invention.

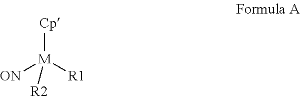

Formula B shows one particular, but non-limiting, embodiment of this molecule, wherein R1 is hydrogen (“(e)” in the Summary's description) and R2 is η³-allyl (“(f)” in the Summary's description).

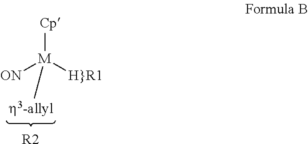

In all embodiments of Formula A, Cp′ is a substituted or unsubstituted η⁵-cyclopentadienyl group, and substituent(s) are independently selected from hydrogen and moieties containing from 1 to 40, preferably 1 to 30, more preferably from 1 to 20, non-hydrogen atoms. The non-hydrogen atoms may include, for example, carbon, nitrogen, oxygen, silicon, halogens, and combinations thereof. These moieties may be selected from hydrocarbyl, arylalkyl, hydrocarbylsilyl, halo-substituted hydrocarbyl, hydrocarbyloxy-substituted hydrocarbyl, hydrocarbylamino-substituted hydrocarbyl, hydrocarbylsilyl-substituted hydrocarbyl, hydrocarbylamino, di(hydrocarbyl)amino, and hydrocarbyloxy moieties, and combinations thereof.

Certain embodiments of the invention are illustrated in the Examples hereinbelow. However, it is found that it may be convenient to prepare the transition metal complex, i.e., the organometallic composition, by reacting as starting materials a halide, such as (η⁵—C₅(CH₃)₅)M(NO)(alkyl)Cl or (η⁵—C₅(CH₃)₅)M(NO)Cl₂, where (M=Mo or W) with an appropriate alkylating agent, such as an organomagnesium or organolithium reagent. Suitable examples of organomagnesium reagents may include (allyl)₂Mg.x(dioxane), (alkyl)₂Mg.x(dioxane), and combinations thereof. These reactants may be contacted either neat or in an inert solvent, such as diethyl ether. In some embodiments the reaction solution is then cooled, by means such as in a liquid nitrogen or dry ice/acetone bath, to crystallize the transition metal complex. In other embodiments steps such as washing, followed by drying under vacuum, and/or use of column chromatography, may be added in order to isolate and/or purify the complex. For laboratory scale purposes, Schlenk tubes or flasks may be particularly useful in order to eliminate the possibility of interaction with air. Further general direction may be found in, for example, Debad, J. D.; Legzdins, P.; Rettig, S. J.; Veltheer, J. E. “Reactivity of the Lewis acids Cp*M(NO)(CH₂CMe₃)Cl [M=molybdenum, tungsten] and related complexes,” Organometallics 1993, 12, 2714-2725.

In general the reaction to form the transition metal complex may be conducted at a temperature ranging from −200 degrees Celsius (° C.) to 50° C., preferably from −80° C. to 25° C.; pressure ranging from ambient (101 kilopascals (kPa)) to 1000 kPa; and time ranging from 0.5 hour (h) to 24 h, preferably from 0.5 h to 3 h. Such may preferably be conducted under an inert atmosphere, such as nitrogen. However, those skilled in the art will be aware of the need and desire to balance acceptable completion times with avoidance of degradation of either reactants or product, and therefore will be able to adjust process parameters suitably, either within or outside of the guideline ranges provided above, in order to accomplish the goals and spirit of the invention.

Once an appropriate transition metal complex has been prepared or obtained, it is ready to be used in a reaction with the selected alkyl-containing compound, under any condition or combination of conditions suitable to result in an olefin, preferably a terminal olefin, via dehydrogenation. Such may, in certain non-limiting embodiments, be carried out neat, preferably in a solution of the alkyl-containing compound to be dehydrogenated, or in alternative embodiments, using another solvent, which may be selected from, for example, any of a variety of perfluorinated organic solvents, such as perfluorobenzene, tetradecafluorohexane, perfluorocyclohexane, and combinations thereof.

In some embodiments the temperature of the reaction may preferably range from 25° C. to 200° C., preferably from 25° C. to 150° C., while pressure may preferably range from 101 kPa to 10,500 kPa, more preferably from 101 kPa to 5,000 kPa, and most preferably from 101 kPa to 3,800 kPa. A time period of from 0.5 h to 100 h is preferred, more preferably from 0.5 h to 24 h.

In some embodiments the temperature of the dehydrogenation reaction may preferably range from 25° C. to 200° C., preferably from 25° C. to 150° C., while pressure may preferably range from 101 kPa to 10,500 kPa, more preferably from 101 kPa to 5,000 kPa, and most preferably from 101 kPa to 3,800 kPa. A time period of from 0.5 h to 100 h is preferred, more preferably from 0.5 h to 24 h.

While the temperatures, pressures and times of the previous paragraph may be generally employed, there are also preferences with respect to the identity of the metal used in the transition metal complex. Where M is tungsten, the dehydrogenation temperature is preferably from 60° C. to 200° C. Conversely, where M is molybdenum, a preferred temperature range is from 25° C. to 150° C. As with preparation of the transition metal complex, those skilled in the art will be aware of the need to balance efficiency of production with potential degradation of reactants, products, or both.

The present dehydrogenation process offers certain advantages that may enhance its adaptability to commercial practice. In certain embodiments this dehydrogenation may be carried out on an alkyl-containing compound that has not been previously dried, i.e., on a “wet” substrate. As the term is used herein, “undried” refers to an alkyl-containing compound having a water content that is 0.01 weight percent (wt %) or higher, while “dried” refers to an alkyl-containing compound having a water content that is less than 0.01 wt %, preferably less than 0.001 wt %, and more preferably less than 0.0001 wt %. Use of a “wet” substrate significantly decreases production cost while it increases convenience. As previously noted, it may also be carried out on a neat alkyl-containing compound, or on an alkyl-containing compound in solution with a perfluorinated solvent. As noted hereinabove, it may be carried out under air and does not require a dedicated and more expensive atmosphere such as nitrogen. Of particular advantage is the fact that in certain embodiments it may be conducted without use of a sacrificial olefin to drive the equilibrium by removing hydrogen.

### EXAMPLE 1

### I. Preparation of (η⁵—C₅(CH₃)₅)W(NO)(CH₂C(CH₃)₃)₂ (Transition Metal Complex (TMC) 1)

In a glove box, a Schlenk flask is charged with (η⁵—C₅(CH₃)₅)W(NO)Cl₂ (8.537 grams (g), 20.33 millimoles (mmol) and tetrahydrofuran (THF) (approximately (ca.) 150 milliliters (mL)), then cooled to −78° C. in a dry ice/acetone bath. A second Schlenk flask is charged in the glove box with the reagent Mg(CH₂C(CH₃)₃)₂ (Titre: 122 grams per mole (g/mol), 2.473 g, 20.27 mmol) and THF (ca. 50 mL), and this mixture is added slowly via cannula to the original Schlenk flask. After the addition is complete, the Schlenk flask is removed from the cold bath, and its contents are stirred at room temperature for 0.5 h to obtain a dark purple mixture. The THF is removed in vacuo, and the contents are transferred with diethyl ether (Et₂O) (6 mL×25 mL) to a second flask at −78° C. which is charged with Mg(CH₂C(CH₃)₃)₂ (Titre: 122 g/mol, 2.531 g, 20.75 mmol), Et₂O (ca. 25 mL), and a magnetic stir bar. The flask is then removed from the dry ice/acetone bath, and its contents are stirred at room temperature for 2.5 h to obtain a burgundy-colored mixture that is transferred directly to the top of a basic alumina column (3 centimeters (cm)×6 cm) made up in diethyl ether (Et₂O). Elution of the column with Et₂O develops a pink-red band that elutes as a red solution. Removing the solvent from the eluate in vacuo affords (η⁵—C₅(CH₃)₅)W(NO)(CH₂C(CH₃)₃)₂ (TMC 1) as a burgundy-colored solid (6.767 g, 68 percent (%) yield). See also, e.g., Bau, R.; Mason, S. A.; Patrick, B. O.; Adams, C. S.; Sharp, W. B.; Legzdins, P. “Alpha-Agostic Interactions in Cp*W(NO)(CH₂CMe₃)₂ and Related Nitrosyl Complexes,” Organometallics 2001, 20, 4492-4501, which includes characterization data of a material corresponding to TMC 1.

### II. Dehydrodegenations Using (η⁵—C₅(CH₃)₅)W(NO)(CH₂C(CH₃)₃)₂ (TMC 1)

(A) Dehydrogenation of Dried n-Pentane.

In a glove box a glass Schlenk flask is charged with (η⁵—C₅(CH₃)₅)W(NO)(CH₂C(CH₃)₃)₂ (TMC 1) (0.105 g, 0.214 mmol) and n-pentane (ca. 15 mL). The Schlenk flask is sealed with a KONTES™ (KONTES™ is a trademark of Kimble Kontes Asset Mgmt. Inc.) greaseless stopcock, and then its contents are heated for 15 h at 81° C. to produce a brown mixture. Analysis of the crude reaction mixture by ¹H NMR spectroscopy reveals the presence of 1-pentene and 2-pentene as well as three isomers of (η⁵—C₅(CH₃)₅)W(NO)(H)(η³—C₅H₉) (TMC 8). The ratio of terminal to internal pentene is 76:24. The volatile components of the reaction mixture are removed in vacuo to obtain (η⁵—C₅(CH₃)₅)W(NO)(H)(η³—C₅H₉) (TMC 8) as a brown solid residue (0.083 g, 93% yield). See also, e.g., Baillie, R. A.; Tran, T.; Lalonde, K. M.; Tsang, J. Y. K.; Thibault, M. E.; Patrick, B. O.; Legzdins, P. “Factors Influencing the Outcomes of Inter-molecular CH Activations of Hydrocarbons Initiated by CpW(NO)(CH₂CMe₃)(η³-Allyl) Complexes (Cp=η⁵—C₅Me₅ (Cp*), η⁵—C₅Me₄H (Cp′)),” Organometallics 2012, 31, 1055-1067, which includes characterization data for three isomers of a material corresponding to TMC 8.

(B) Dehydrogenation of Undried n-Pentane.

Replicate Example 1(II)(A), but replace dried n-pentane with undried n-pentane (water content greater than 0.01 wt %) and carry out dehydrogenation at 81° C. for 61 h. ¹H NMR spectroscopic analysis of the crude reaction mixture reveals a selectivity of 1-pentene to 2-pentene of 67:33.

(C) Dehydrogenation of Undried n-Hexane.

In a glove box, a glass Schlenk flask is charged with (η⁵—C₅(CH₃)₅)W(NO)(CH₂C(CH₃)₃)₂ (TMC 1) (0.172 g, 0.350 mmol) and undried n-hexane (ca. 15 mL). The Schlenk flask is then sealed with a KONTES™ greaseless stopcock, and then its contents are heated for 16 h at 81° C. to produce a dark brown mixture. Analysis of the crude reaction mixture by ¹H NMR spectroscopy reveals the presence of isomers of hexene and isomers of (η⁵—C₅(CH₃)₅)W(NO)(H)(η³—C₆H₁₁) (TMC 9). The ratio of 1-hexene to the internal isomers of hexene is 65:35. The volatile components of the reaction mixture are removed in vacuo and TMC 9 is obtained as a brown solid residue (0.148 g, 98% yield). TMC 9 is characterized by ¹H NMR spectroscopy, IR spectroscopy, and mass spectrometry.

(D) Dehydrogenation of Undried n-Octane.

Replicate Example 1(II)(A), but replace dried n-pentane with undried n-octane (ca. 15 mL), use (η⁵—C₅(CH₃)₅)W(NO)(CH₂C(CH₃)₃)₂ (TMC 1) (0.094 g, 0.248 mmol), and heat the contents of the flask for 90 h at 90° C. A dark brown final mixture is obtained, and an analysis of the mixture by ¹H NMR spectroscopy reveals the ratio of 1-octene to all internal isomers of octene is 35:65.

(E) Dehydrogenation of Undried Ethylbenzene.

In a glove box, a sample of (η⁵—C₅(CH₃)₅)W(NO)(CH₂C(CH₃)₃)₂ (TMC 1) (0.093 g, 0.19 mmol) is dissolved in undried ethylbenzene (ca. 5 mL) and transferred to a Schlenk flask, which is then sealed with a greaseless KONTES™ stopcock. The flask is placed in an 81° C. ethylene glycol bath for 16 h to produce a dark purple mixture. Analysis of the crude reaction mixture by ¹H NMR spectroscopy reveals the presence of styrene.

(F) Dehydrogenation of Undried Propylbenzene.

Replicate Example 1(II)(E), using (η⁵—C₅(CH₃)₅)W(NO)(CH₂C(CH₃)₃)₂ (TMC 1) (0.142 g, 0.289 mmol) and undried propylbenzene (ca. 5 mL) in place of ethylbenzene. The mixture is heated using an ethylene glycol bath at 81° C. for 16 h whereupon the solution changes from burgundy to dark purple in color. An analysis of the crude reaction mixture is performed by ¹H NMR spectroscopy to reveal the presence of trans-6-methylstyrene.

### EXAMPLE 2

### I. Preparation of (η₅—C₅(CH₃)₅)Mo(NO)(CH₂C(CH₃)₃)₂ (TMC 2)

Replicate Example 1(1), preparation of TMC 1, but use (η⁵—C₅(CH₃)₅)Mo(NO)Cl₂ (3.848 g, 11.58 mmol) and Mg(CH₂C(CH₃)₃)₂ (Titre: 142 g/mol, 1.643 g, 11.57 mmol), to form (η⁵—C₅(CH₃)₅)Mo(NO)—(CH₂C(CH₃)₃)Cl as a dark purple solid. The second Schlenk flask is charged with Mg(CH₂C(CH₃)₃)₂ (Titre: 142 g/mol, 1.668 g, 11.75 mmol) to obtain (η⁵—C₅(CH₃)₅)Mo(NO)(CH₂C(CH₃)₃)₂ (TMC 2) as a burgundy solid (2.993 g, 63% yield). See also, e.g., Pamplin, C. B.; Legzdins, P., ibid., which includes characterization data of a material corresponding to TMC 2.

### II. Dehydrogenations Using (η⁵—C₅(CH₃)₅)Mo(NO)(CH₂C(CH₃)₃)₂ (TMC 2)

(A) Dehydrogenation of Undried n-Pentane.

In a glove box a glass Schlenk flask is charged with (η⁵—C₅(CH₃)₅)Mo(NO)(CH₂C(CH₃)₃)₂ (TMC 2) (0.160 g, 0.397 mmol) and undried n-pentane (ca. 15 mL) and then sealed with a KONTES™ greaseless stopcock. The Schlenk flask is placed in an ethylene glycol bath maintained at 40° C. for 4 days (d) to obtain a dark brown mixture. Volatiles are removed by distillation from the final mixture and analyzed by ¹H NMR spectroscopy which reveals the formation of 1-pentene exclusively.

(B) Dehydrogenation of Undried n-Hexane.

Replicate Example 2(II)(A), using (η⁵—C₅(CH₃)₅)Mo(NO)(CH₂C(CH₃)₃)₂ (TMC 2) (0.081 g, 0.20 mmol) and undried n-hexane (ca. 15 mL), stir the mixture at room temperature for 16 h to obtain a dark brown mixture. Analysis of the reaction mixture by ¹H NMR spectroscopy reveals the formation of 1-hexene exclusively.

(C) Dehydrogenation of Undried n-Octane.

Replicate Example 2(II)(A), using (η⁵—C₅(CH₃)₅)Mo(NO)(CH₂C(CH₃)₃)₂ (TMC 2) (0.133 g, 0.327 mmol) and undried n-octane (ca. 18 mL), and the flask is then sealed with a KONTES™ greaseless stopcock. The burgundy-colored mixture is stirred at room temperature for 2.5 d to obtain a dark brown mixture. Analysis of this mixture by ¹H NMR spectroscopy reveals the exclusive formation of 1-octene.

### EXAMPLE 3

### I. Preparation of (η⁵—C₅H₅)W(NO)(CH₂C(CH₃)₃)₂ (TMC 3)

Replicate Example 1(1), preparation of TMC 1, but use (η⁵—C₅H₅)W(NO)Cl₂ (2.721 g, 7.78 mmol), Mg(CH₂C(CH₃)₃)₂ (Titre: 115 g/mol, 0.855 g, 7.44 mmol), to form (η⁵—C₅H₅)W(NO)(CH₂C(CH₃)₃)Cl as a purple solid. The second Schlenk flask is charged with Mg(CH₂C(CH₃)₃)₂ (Titre: 115 g/mol, 0.850 g, 7.39 mmol) to obtain (η⁵—C₅H₅)W(NO)(CH₂C(CH₃)₃)₂ (TMC 3) as a dark red solid (0.850 g, 27% yield). See also, e.g., Legzdins, P.; Rettig, S. J.; Sanchez, L. “Organometallic nitrosyl chemistry. 37. Synthesis, characterization, and some chemical properties of unusual 16-electron dialkyl (η⁵-cyclopentadienyl)-nitrosylmolybdenum and tungsten complexes,” Organometallics 1988, 7, 2394-2403, which includes characterization data of a material corresponding to TMC 3.

### II. Dehydrogenation Using (η⁵—C₅H₅)W(NO)(CH₂C(CH₃)₃)₂ (TMC 3)

(A) Dehydrogenation of Undried n-Pentane.

Replicate Example 1(II)(A), using (η⁵—C₅H₅)W(NO)(CH₂C(CH₃)₃)₂ (TMC 3) (0.260 g, 0.617 mmol) and undried n-pentane (ca. 10 mL), and the flask is then sealed with a KONTES™ greaseless stopcock. The Schlenk flask is placed in an ethylene glycol bath maintained at 81° C. for 70 h to obtain a dark brown mixture. ¹H NMR spectroscopic analysis of the crude reaction mixture reveals a selectivity of 1-pentene to 2-pentene of 67:33.

### EXAMPLE 4

### I. Preparation of (η⁵—C₅(CH₃)₄(C₂H₅))W(NO)(CH₂C(CH₃)₃)₂ (TMC 4)

In a glove box, a Schlenk flask is charged with (η⁵—C₅(CH₃)₄(C₂H₅))W(NO)Cl₂ (2.918 g, 6.74 mmol), a light green powder, Mg(CH₂C(CH₃)₃)₂ (Titre: 118 g/mol, 0.794 g, 6.73 mmol), a white powder, and a magnetic stir bar. The mixture is cooled to −196° C. with a liquid nitrogen bath and Et₂O (ca. 150 mL) is added dropwise via cannulation. The Schlenk flask is warmed to room temperature while being stirred for 1 h, resulting in a dark purple solution. A second Schlenk flask is charged with Mg(CH₂C(CH₃)₃)₂ (Titre: 118 g/mol, 0.822 g, 6.966 mmol), which is cooled to −196° C. using a liquid nitrogen bath. The mixture from the first flask is then transferred dropwise via cannulation into the second flask. After the addition the flask is allowed to warm up to room temperature while being stirred for 1 h to produce a dark maroon-colored solution. The mixture is then filtered through CELITE™ (CELITE™ is a trademark of Imerys Minerals California, Inc.) using a porous frit to remove the magnesium salts. The solvent is removed in vacuo to give an oily residue. The sample is re-dissolved in pentane and chromatographed on a basic alumina column (3.5 cm×0.5 cm). A maroon-colored band is eluted with 3:1 pentane/Et₂O to give a maroon solution. Solvent is removed in vacuo and affords (η⁵—C₅(CH₃)₄(C₄H₅))W(NO)(CH₂C(CH₃)₃)₂ (TMC 4) as maroon-colored needles (1.042 g, 31% yield). Characterization data confirming TMC 4 is obtained via ¹H and ¹³C NMR spectroscopy, IR spectroscopy and mass spectrometry.

### II. Dehydrogenation Using (η⁵—C₅(CH₃)₄(C₂H₅))W(NO)(CH₂C(CH₃)₃)₂ (TMC 4)

(A) Dehydrogenation of Undried n-Pentane.

Replicate Example 1(II)(A), using (η⁵—C₅(CH₃)₄(C₂H₅))W(NO)(CH₂C(CH₃)₃)₂ (TMC 4) (0.100 g, 0.198 mmol) and undried n-pentane (ca. 10 mL), and the flask is then sealed with a KONTES™ greaseless stopcock. The Schlenk flask is placed in an ethylene glycol bath maintained at 81° C. for 61 h to obtain a dark brown mixture. ¹H NMR spectroscopic analysis of the crude reaction mixture reveals a selectivity of 1-pentene to 2-pentene of 76:24.

### EXAMPLE 5

### I. Preparation of (η⁵—C₅(CH₃)₅)W(NO)(CH₂C(CH₃)₃)(CH₂Si(CH₃)₃)(TMC 5)

Replicate Example 1(1), preparation of TMC 1, but use (η⁵—C₅(CH₃)₅)W(NO)Cl₂ (3.289 g, 7.829 mmol), Mg(CH₂C(CH₃)₃)₂ (Titre: 130 g/mol, 1.013 g, 7.79 mmol), to form (η⁵—C₅(CH₃)₅)W(NO)—(CH₂C(CH₃)₃)Cl as a dark purple solid. The second Schlenk flask is charged with Mg(CH₂Si(CH₃)₃)₂ (Titre: 200 g/mol, 1.582 g, 7.91 mmol) to obtain (η⁵—C₅(CH₃)₅)W(NO)(CH₂C(CH₃)₃)(CH₂Si(CH₃)₃) (TMC 5) as a purple solid (2.148 g, 54% yield). Characterization data confirming TMC 5 is obtained via ¹H and ¹³C NMR spectroscopy, IR spectroscopy and mass spectrometry.

### II. Dehydrogenation Using (η⁵—C₅(CH₃)₅)W(NO)(CH₂C(CH₃)₃)(CH₂Si(CH₃)₃) (TMC 5)

(A) Dehydrogenation of Undried n-Pentane.

Replicate Example 1(II)(A), using (η⁵—C₅(CH₃)₅)W(NO)(CH₂C(CH₃)₃)(CH₂Si(CH₃)₃) (TMC 5) (0.057 g, 0.112 mmol) and undried n-pentane (ca. 10 mL), and the flask is then sealed with a KONTES™ greaseless stopcock. The Schlenk flask is placed in an ethylene glycol bath maintained at 120° C. for 20 h to obtain a dark brown mixture. ¹H NMR spectroscopic analysis of the crude reaction mixture reveals a selectivity of 1-pentene to 2-pentene of 70:30.

### EXAMPLE 6

### I. Preparation of (η⁵—C₅(CH₃)₅)W(NO)(H)(η³—CH₂CHCH(C₆H₅)) (TMC 6)

In a glove box, a Schlenk flask is charged with (η⁵—C₅(CH₃)₅)W(NO)Cl₂ (5.000 g, 11.90 mmol) and a magnetic stir bar. A second Schlenk flask is charged with Mg(CH₂CH═CH(C₆H₅))₂ (Titre: 218 g/mol, 2.592 g, 11.90 mmol) and a magnetic stir bar. On a Schlenk line under argon, dry THF (ca. 100 mL each) is cannulated into each Schlenk flask, and each mixture is stirred until all solid material dissolved. Both Schlenk flasks are then placed into a dry ice/acetone bath (−78° C.) while stirring of their contents is maintained. Once cold, the contents of the second Schlenk flask are cannulated slowly into the first Schlenk flask. The resulting mixture is then removed from the cold bath and is allowed to reach room temperature while being stirred for 1 h. The first Schlenk flask is then placed back into the dry ice/acetone bath, and its contents are cooled to −78° C. A solution of lithium borohydride (LiBH₄) in THF (2.0 M, 6.5 mL, 13 mmol) is added to the Schlenk flask in a dropwise fashion. The mixture develops a strong red-brown color, and it is removed from the cold bath and allowed to reach room temperature while being stirred for 3 h. Removal of the THF in vacuo leaves behind a reddish brown oily residue. On the bench top, this residue is re-dissolved in Et₂O and the mixture is then filtered through CELITE™ using a porous frit to remove the magnesium salts. The ether layer is dark reddish brown in color at this stage, and it is reduced in volume in vacuo to obtain a concentrated solution of the crude product. Purification is performed over neutral alumina, with the chromatography column (10 cm×2.5 cm) being prepared with hexanes. A yellow band is eluted with a gradient of 0-50% Et₂O in hexane to give a yellow solution. Solvent is removed in vacuo and affords (η⁵—C₅(CH₃)₅)W(NO)(H)(η³—CH₂CHCH(C₆H₅)) (TMC 6) as a yellow powder (1.1145 g, 20% yield). Characterization data for TMC 6 is obtained, including ¹H and ¹³C NMR spectroscopy, IR spectroscopy and mass spectrometry.

### II. Dehydrogenation Using (η⁵—C₅(CH₃)₅)W(NO)(H)(η³—CH₂CHCH(C₆H₅)) (TMC 6)

(A) Dehydrogenation of Undried n-Pentane.

Replicate Example 1(II)(A), using (η⁵—C₅(CH₃)₅)W(NO)(H)(η³—CH₂CHCH(C₆H₅)) (TMC 6) (0.0844 g, 0.180 mmol) and undried n-pentane (ca. 15 mL), and the flask is then sealed with a KONTES™ greaseless stopcock. The Schlenk flask is placed in an ethylene glycol bath maintained at 81° C. for 5 d, subsequently placed in an ethylene glycol bath maintained at 120° C. for 12 h to obtain a dark brown mixture. ¹H NMR spectroscopic analysis of the crude reaction mixture reveals a selectivity of 1-pentene to 2-pentene of 61:39.

### EXAMPLE 7

### I. Preparation of (η⁵—C₅(CH₃)₅)W(NO)(H)(η³—CH₂CHC(CH₃)₂) (TMC 7)

In a glove box, a glass Schlenk flask is charged with (η⁵—C₅(CH₃)₅)W(NO)Cl₂ (4.210 g, 10.00 mmol) and THF (ca. 60 mL), and then cooled in a dry ice/acetone bath (−78° C.). A second Schlenk flask is charged with Mg(CH₂CHC(CH₃)₂)₂ (Titre: 128 g/mL, 1.314 g, 10.00 mmol) and THF (ca. 50 mL), and then its contents are transferred to the first flask dropwise via cannula. Following this addition, the reaction mixture is allowed to warm to room temperature and is then stirred for 1 h to obtain a yellow-brown mixture. The Schlenk flask is again cooled again to −78° C., and LiBH₄ in THF (2.0 M, 5.0 mL, 10.00 mmol) is added slowly via syringe. The contents of the Schlenk flask are warmed to room temperature and stirred for 3 h to obtain a brown mixture. The THF is removed from this mixture in vacuo, and the residue is taken up in Et₂O (ca. 50 mL) and washed with H₂O (3×50 mL). The Et₂O layer is filtered through a glass frit, and then the solvent is removed from the filtrate under reduced pressure to obtain a brown solid. The solid is re-dissolved in Et₂O, and the solution is transferred to the top of a basic alumina column (3 cm×5 cm) that has been made up in hexanes. A yellow band is developed with Et₂O as eluent and is collected as a yellow eluate. The Et₂O is removed from the eluate in vacuo, and the residue is washed with cold pentane to obtain (η⁵—C₅(CH₃)₅)W(NO)(H)(η³—CH₂CHC(CH₃)₂) (TMC 7) as a yellow solid (2.866 g, 65% yield).

### II. Dehydrogenation Using (η⁵—C₅(CH₃)₅)W(NO)(H)(η³—CH₂CHC(CH₃)₂) (TMC 7)

(A) Dehydrogenation of Dried n-Pentane

Replicate Example 1(II)(A), using (η⁵—C₅(CH₃)₅)W(NO)(H)(η³—CH₂CHC(CH₃)₂) (TMC 7) (0.079 g, 0.188 mmol) and dried n-pentane (ca. 10 mL), and the flask is then sealed with a KONTES™ greaseless stopcock. The Schlenk flask is placed in an ethylene glycol bath maintained at 81° C. for 3 d to obtain a dark brown mixture. ¹H NMR spectroscopic analysis of the crude reaction mixture reveals a selectivity of 1-pentene to 2-pentene of 70:30.

### EXAMPLE 8

### I. Preparation of (η⁵—C₅(CH₃)₅)W(NO)(H)(η³—C₅H₉) (TMC 8)

In a glove box, a Schlenk flask is charged with (η⁵—C₅(CH₃)₅)W(NO)(CH₂C(CH₃)₃)₂ (TMC 1) (1.065 g, 2.17 mmol) and dried n-pentane (ca. 300 mL) and then sealed with a KONTES™ stopcock. The Schlenk flask is placed in an ethylene glycol bath maintained at 81° C. for 16 h. The solution changes from burgundy to brown in color. Once removed from the bath, all of the solvent and volatiles are removed from the mixture in vacuo. The solid is re-dissolved in Et₂O, and the solution is transferred to the top of a basic alumina column (3 cm×10 cm) made up in hexanes. A yellow band is developed with Et₂O as eluent and is collected as a yellow eluate. The Et₂O is removed from the eluate in vacuo, and the residue is washed with cold pentane to obtain (η⁵—C₅(CH₃)₅)W(NO)(H)(η³—C₅H₉) (TMC 8) as a yellow solid (0.331 g, 36% yield). See also, e.g., Baillie, R. A.; Tran, T., et al., ibid., which includes characterization data corresponding to a TMC 8 material.

### II. Dehydrogenation with (η⁵—C₅(CH₃)₅)W(NO)(H)(η³—C₅H₉) (TMC 8)

(A) Dehydrogenation of Undried Propylbenzene.

In a glove box a Schlenk flask is charged with (η⁵—C₅(CH₃)₅)W(NO)(H)(η³—C₅H₉) (TMC 8) and undried propylbenzene (ca. 10 mL) and then sealed with a KONTES™ greaseless stopcock. The Schlenk flask is placed in an ethylene glycol bath maintained at 71° C. for 90 h. An analysis of a sample from the final reaction mixture is performed by ¹H NMR spectroscopy which confirms the formation of trans-6-methylstyrene.

### EXAMPLE 9

### I. Preparation of (η⁵—C₅H₄CH(CH₃)₂)W(CH₂C(CH₃)₃)₂ (TMC 10)

A standard solution of (η⁵—C₅H₄CH(CH₃)₂)W(NO)(CO)₂ in Et₂O (9.5 mL) is prepared under anaerobic conditions. An aliquot (0.3 mL) is extracted via syringe and combined with a known amount of ferrocene (0.0216 g, 0.116 mmol) to determine the concentration of the (η⁵—C₅H₄CH(CH₃)₂)W(NO)(CO)₂ (11.4 mmol) via integration of the ¹H NMR spectrum of the sample in benzene-d₆. In the glovebox, a Schlenk flask is charged with PCl₅ (2.350 g, 11.3 mmol). The contents of the Schlenk are cooled to −196° C. with a liquid nitrogen bath and Et₂O (ca. 150 mL) is added via cannulation, followed by addition of the orange standard solution. The Schlenk flask is warmed to room temperature while being stirred for 2 h, resulting in a dark green solution of (η⁵—C₅H₄CH(CH₃)₂)W(NO)Cl₂. In the glovebox, a second Schlenk flask is charged with Mg(CH₂C(CH₃)₃)₂ (Titre: 170.5 g/mol, 1.955 g, 11.5 mmol) and Et₂O (ca. 100 mL). The mixture is then transferred dropwise via cannulation into the reaction Schlenk flask with (η5-C₅H₄CH(CH₃)₂)W(NO)Cl₂ placed into a dry ice/acetone bath (−78° C.). The flask is then removed from the bath, and its contents are stirred at room temperature for 30 minutes (min) to produce a dark brown solution. The mixture is then filtered through CELITE™ using a porous frit to remove the magnesium salts. The sample is transferred into another reaction Schlenk flask charged with Mg(CH₂C(CH₃)₃)₂ (Titre: 170.5 g/mol, 1.950 g, 11.4 mmol) and Et₂O (ca. 100 mL) at −78° C. The flask is then removed from the dry ice/acetone bath, and its contents are stirred at room temperature for 30 min to obtain burgundy-colored mixture. After the volume of the reaction mixture is reduced in vacuo, the solution is transferred to the top of a basic alumina column (3 cm×6 cm) made up in Et₂O. A red band is eluted with Et₂O to give a burgundy solution. Solvent is removed in vacuo to afford (η⁵—C₅H₄CH(CH₃)₂)W(CH₂C(CH₃)₃)₂ as a burgundy-colored solid (1.787 g, 3.86 mmol, 34% yield).

Characterization data included ¹H NMR spectroscopy, IR spectroscopy, and mass spectrometry.

### II. Dehydrogenation Using (η⁵—C₅H₄CH(CH₂)W(CH₂CMe₃)₂ (TMC 10)

(A) Dehydrogenation of Undried n-Pentane.

Replicate Example 1(II)(A), using (η⁵—C₅H₄CH(CH₃)₂)W(CH₂C(CH₃)₃)₂ (0.106 g, 0.229 mmol) and undried n-pentane (ca. 10 mL). The Schlenk flask is placed in an ethylene glycol bath maintained at 60° C. for 2 d to obtain a dark brown mixture. ¹H NMR spectroscopic analysis of the crude reaction mixture reveals a selectivity of 1-pentene to 2-pentene of 70:30.

Table 1 includes TMCs 1-10 and shows the dehydrogenation performance data for each, except for TMC 9, which has not been tested in a dehydrogenation.

TABLE 1
Identification of TMCs 1-10 and Dehydrogenation Performance Data for TMCs 1-8 and 10

| TMC # | TMC formula | Dehydrogenation Substrate | Dehydrogenation Yield and Ratio |
|---|---|---|---|
| 1 | (η⁵-C₅(CH₃)₅)W(NO)(CH₂C(CH₃)₃)₂ | n-pentane (dried*) | 1-pentene:2-pentene 76:24 |
| | | n-pentane (undried**) | 1-pentene:2-pentene 67:33 |
| | | n-hexane (undried) | 1-hexene:internal isomers 65:35 |
| | | n-octane (undried) | 1-octene:internal isomers 35:65 |
| | | ethylbenzene (undried) | styrene |
| | | propylbenzene (undried) | trans-β-methylstyrene |
| 2 | (η⁵-C₅(CH₃)₅)Mo(NO)(CH₂C(CH₃)₃)₂ | n-pentane (undried) | 1-pentene |
| | | n-hexane (undried) | 1-hexene |
| | | n-octane (undried) | 1-octene |
| 3 | (η⁵-C₅H₅)W(NO)(CH₂C(CH₃)₃)₂ | n-pentane(undried) | 1-pentene:2-pentene 67:33 |
| 4 | (η⁵-C₅(CH₃)₄(C₂H₅))W(NO)(CH₂C(CH₃)₃)₂ | n-pentane (undried) | 1-pentene:2-pentene 72:28 |
| 5 | (η⁵-C₅(CH₃)₅)W(NO)(CH₂C(CH₃)₃)(CH₂Si(CH₃)₃) | n-pentane (undried) | 1-pentene:2-pentene 70:30 |
| 6 | (η⁵-C₅(CH₃)₅)W(NO)(H)(η³-CH₂CHCH(C₆H₅)) | n-pentane (undried) | 1-pentene:2-pentene 61:39 |
| 7 | (η⁵-C₅(CH₃)₅)W(NO)(H)(η³-CH₂CHC(CH₃)₂) | n-pentane (dried) | 1-pentene:2-pentene 70:30 |
| 8 | (η⁵-C₅(CH₃)₅)W(NO)(H)(η³-C₅H₉) | propylbenzene (undried) | trans-β-methylstyrene |
| 9 | (η⁵-C₅(CH₃)₅)W(NO)(H)(η³-C₆H₁₁) | n/a | n/a |
| 10 | (η⁵-C₅H₄CH(CH₃)₂)W(CH₂C(CH₃)₃)₂ | n-pentane (undried) | 1-pentene:2-pentene 70:30 |

*dried indicates that the alkyl containing compound contains less than 0.0001 wt % of water prior to reaction.
**undried indicates that the alkyl containing compound contains from 0.0001 to 0.01 wt % of water prior to reaction.
n/a indicates not tested in a dehydrogenation

1. A process for a dehydrogenation, comprising:
reacting an alkyl-containing compound and a transition metal complex having the composition
Cp′M(NO)(R1)(R2)
wherein Cp′ is a substituted or unsubstituted η⁵-cyclopentadienyl group, wherein the substituents are independently selected from hydrogen and moieties containing from 1 to 40 non-hydrogen atoms selected from hydrocarbyl, arylalkyl, hydrocarbylsilyl, halo-substituted hydrocarbyl, hydrocarbyloxy-substituted hydrocarbyl, hydrocarbylamino-substituted hydrocarbyl, hydrocarbylsilyl-substituted hydrocarbyl, hydrocarbylamino, di(hydrocarbyl)amino, and hydrocarbyloxy moieties, and combinations thereof; and
wherein M is selected from W and Mo; and
wherein R1 and R2 are each independently selected from
(a) CH₂C(CH₃)₃;
(b) CH₂Si(CH₃)₃;
(c) CH₂(C₆H₅);
(d) CH₃;
(e) hydrogen; and
(f) η³-allyl;
provided that if R1 is hydrogen, R2 is η³-allyl;
under conditions such that the alkyl-containing compound is converted to an olefin.

2. The process of claim 1 wherein η³-allyl is an allyl ligand selected from:
(a) η³—CₙH₍₂ₙ₋₁₎;
(b) η³—CH₂CH(CH₃)₂;
(c) η³—CH₂CHCHSi(CH₃)₃;
(d) η³—CH₂CHCH(C₆H₅); and
(e) η³—CH(C₆H₅)CHCH(C₆H₅);
wherein n is an integer from 3 to 10.

3. The process of claim 1 wherein the conditions include a temperature ranging from 25° C. to 200° C.; a pressure ranging from 101 kilopascals to 10,500 kilopascals; and a time ranging from 0.5 hour to 100 hours.

4. The process of claim 3 wherein the pressure ranges from 101 kilopascals to 5,000 kilopascals; and wherein when M is tungsten, the temperature ranges from 60° C. to 200° C., and wherein when M is molybdenum, the temperature ranges from 25° C. to 150° C.

5. The process of claim 1 wherein the alkyl-containing compound has a carbon number ranging from 2 to 20 carbons.

6. The process of claim 1 wherein the alkyl-containing compound contains no more than 0.01 weight percent of water prior to the reaction.

7. The process of claim 1
wherein the olefin includes at least a portion of one or more terminal olefins.

8. The process of claim 1 wherein the transition metal complex is selected from the group consisting of
(a) (η⁵—C₅(CH₃)₅)W(NO)(CH₂C(CH₃)₃)₂;
(b) (η⁵—C₅(CH₃)₅)Mo(NO)(CH₂C(CH₃)₃)₂;
(c) (η⁵—C₅H₅)W(NO)(CH₂C(CH₃)₃)₂;
(d) (η⁵—C₅(CH₃)₄(C₂H₅))W(NO)(CH₂C(CH₃)₃)₂;
(e) (η⁵—C₅(CH₃)₅)W(NO)(CH₂C(CH₃)₃)(CH₂Si(CH₃)₃);
(f) (η⁵—C₅(CH₃)₅)W(NO)(H)(η³—CH₂CHCH(C₆H₅));
(g) (η⁵—C₅(CH₃)₅)W(NO)(H)(η³—CH₂CHC(CH₃)₂);
(h) (η⁵—C₅(CH₃)₅)W(NO)(H)(η³—C₅H₉);
(i) (η⁵ C₅H₄CH(CH₃)₂)W(CH₂C(CH₃)₃)₂; and
(j) combinations thereof.
